(19) United States
(12) Patent Application Publication
(10) Pub. No.: US 2009/0248158 A1
(43) Pub. Date: Oct. 1, 2009

(54) COMBINED FIXATION BONE IMPLANT

(76) Inventors: Antonio Manuel Godinho Completo, Gafanha Da Nazare (PT); Jose Antonio de Oliveira Simoes, Albergaria-A-Velha (PT); Fernando Manuel Pereira Da Fonseca, Coimbra (PT)

Correspondence Address: ARENT FOX LLP, 1675 BROADWAY, NEW YORK, NY, 10019, US

(21) Appl. No.: 12/158,609

(22) PCT Filed: Dec. 20, 2006
(86) PCT No.: PCT/IB2006/054979

§ 371 Date: Nov. 19, 2008

(30) Foreign Application Priority Data
Dec. 21, 2005 (PT) 103404

(51) Int. Cl. A61F 2/28; A61B 17/58

(52) U.S. Cl. 623/16.11; 606/93

(57) ABSTRACT

The present invention relates to a combined fixation bone implant consisting on a (metallic or non-metallic) implant which, due to its geometry, allows its fixation to the bone tissue in two different and simultaneous ways.

In view of its characteristics, it allows that part of its superficial area (coated with any type of material or structure that may stimulate the adhesion or bone integration of the bone tissue) remains in direct contact with the bone tissue and the other part is fixed through the bone cement between the implant and the bone tissue.

The process of the implantation on the bone, as well as the retrieval of the implant in case of revision (replacement), minimises the bone loss.

### OBJECT OF THE INVENTION

The present invention relates to a combined fixation implant which discloses, as a concept, a mixed or combined fixation of the implant to the bone.

### BACKGROUND OF THE INVENTION

Current available techniques to fix bone implants are summed up to two great families: the so-called cemented and cementless implants, also known as “press-fit” implants.

The great advantage of cemented implants (the whole implant is coated with a cement layer) is its immediate post-surgery stability. The patient is able to put strain on the implant.

Furthermore, considering that bone cement comprises antibiotics, this allows for a reduction of infection rates in this type of implant.

Some disadvantages however are related to the use of this conventional type of implant, such as the need for a bone removal larger than the size of the implant to lodge the cement layer.

The most common disadvantage among medical staff is the great difficulty in extracting this type of implant whenever it is necessary to make a revision (replacement), which is always related to a great bone loss or bone fracture during the surgical removal process.

In what concerns cementless implants, their disadvantage is their fixation devices which, depending on the surface texture type, may provide a greater or lesser adhesion (or bone integration) of bone tissue to the implant.

The fixation resistance is expectedly minor.

### SUMMARY OF THE INVENTION

This type of implant allows the implant to have zones of direct contact with the bone tissue and zones where the junction of same to the bone is achieved through the bone cement (usually polymethylmetacrilate—PMMA) contained therein.

The outer surface of the implant has a specific geometric shape which allows a direct contact with the bone as well as the insertion of bone cement between the implant and the bone.

The zones where there is a direct contact with the bone are the outer surfaces of the implant; the zones with bone cement are those where there is a cavity in the surface of the implant, which allows the insertion of bone cement.

The geometric shapes of the implant cavities, as well as their arrangement, will depend on the type of implant and the respective purpose (knee, hip, elbow, ankle, finger, shoulder prosthesis, etc).

Each type of application will require a certain type of cavity shape and arrangement on the surface of the implant, which will depend on the biomechanics of the joint (external geometry of the implant, base material, coating material(s), muscle, ligament and contact strength).

The bone cement will reach the cavities of the implant through a set of internal implant canals, where the cement is inserted under pressure through a hole on the implant.

The amount of cement is limited by the geometry of the cavity both on the implant and bone.

In order to complete the description of the present invention and for a better understanding of its characteristics, a set of drawings is provided, being part of the present description in an illustrative and non-limitative manner:

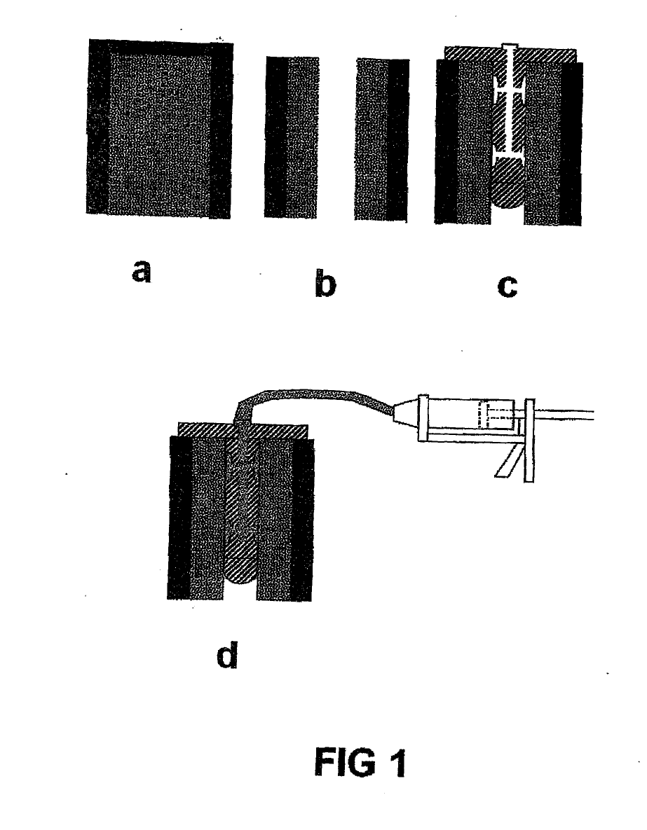

FIG. 1—represents the schematic sequence of the fixation process of the combined fixation implant which is the object of the present invention wherein:
a) shows the scheme of the bone
b) b) shows the scheme of the bone cut for the insertion of the implant
c) c) shows the scheme of the insertion of the combined fixation implant
d) shows the bone cement filling device.

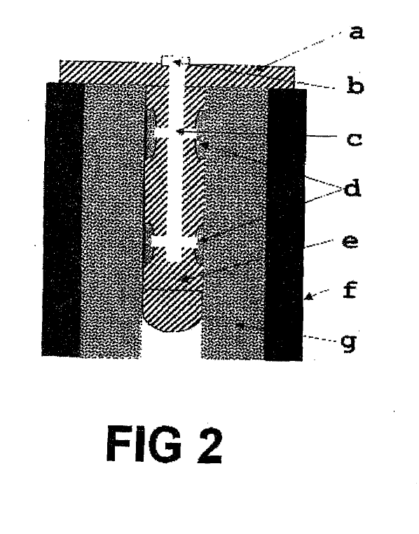

FIG. 2—represents a schematic example of the tibial component of a total knee arthroplasty with a combined fixation rod wherein:
e) shows the tibial plate of the total knee prostehsis;
f) shows the cement filling hole;
g) shows the bone cement circuit;
d) shows the bone cement between the superficial cavity of the implant and the bone;
e) shows the fixation implant;
f) shows the cortical bone of the proximal tibia;
g) shows the cancellous bone of the proximal tibia;

On the same implant surfaces it is possible to have zones of direct contact with the bone and zones where the implant bone junction is materialised through the bone cement.

This concept allows for the combination of the two types of fixation which are currently prevailing in bone implants and available on the market (cemented implants and cementless implants, commonly known as “press fit” implants).

Nowadays, the implants available on the market allow their fixation through a direct contact of all implant surface with the bone (cementless implant), or else implants where their fixation to the bone is achieved through a cement layer (mantle) surrounding all implant surface (cemented implant).

Thus, this new concept of implant (combined fixation) combines the advantages of the two prevailing fixation types on the market, at the same time reducing most of the disadvantages of both fixation types.

### Reduction of Bone Evasion for Fixing the Implant.

Bone cement will be deposited in the existing cavities between the implant and the bone wall, considering that it arrives from the inside of the implant through the existing inner canals. Thus, the required gap for the placement of the implant is limited to the outer shape and size of the implant, being therefore unnecessary to make a larger hole for the placement of the cement mantle, as it usually occurs in cemented implants which require a cavity size larger than the size of the implant for the deposit of the cement layer. In this type of concept there is no cement mantle. With regard to cementless implants (direct implant-bone connection), it also allows a reduction on the bone evasion, considering that this type of cementless fixation requires larger and wider implants to guarantee their stability which results only from the contact with the bone.

Therefore, this type of implant is innovative in limiting the cavity made on the bone for its fixation, when compared to the two types of implants available on the market.

Minimisation of damage caused by the removal of the implant in case of revision (replacement) need

The cement being deposited within the cavities on the surface of the implant, in case there is a need for a revision due to several reasons (infection, implant loss, fracture, etc.) allows its easy removal, since the cement will be completely removed from the bone with the implant retrieval which will carry bone cement on the inside, thus limiting bone damage.

This is an important innovation regarding the fully cemented implants available on the market.

When removing a conventional cemented implant it is necessary to brake and remove all the remaining cement stuck to the bone, considerably damaging it.

Such procedure also implies an extended anaesthesia for the patient, because it is essential to remove all particles of bone cement from the cavity where the implant was lodged for a more effective revision.

The incomplete retrieval of these “debris” may cause some of them. to “migrate” towards a tribologic contact area, accelerating the early erosion of the artificial joint.

The stability of the implant on the bone is assured by the direct contact of the latter to the bone, which is considered to be a long-term stability factor.

On the other hand, the simultaneous fixation with cement in its cavities guarantees an immediate post-surgery stability and avoids excessive micro-movements of the implant, which may endanger bone restructuring on the cementless areas of the implant.

This type of implant, for having less geometral stiffness when compared to an identical conventional implant, will minimise the “stress shielding” phenomenon which leads to the reabsorption of bone tissue.

The fact of the cemented areas being perfectly limited by the implant cavities allows that during the cement healing process (exothermal reaction of polymerisation which increases temperature) the possibly developed bone necrosis is limited to said area, unlike what happens with totally cemented implants in which the whole bone extension in contact with the cement mantle is subject to the development of necrosis.

1. Combined fixation bone implant, characterized in that it presents a geometry (FIG. 2 e), endowed with cavities (FIG. 2 d) in the outer surface, whose cavities can have different forms and sizes, being perfectly aligned by side in the axial direction of the implant (FIG. 2 e), and the external surface form and dimension of implant being perfectly identical to the shaped socket in the bone for implant insertion, and the cavities (FIG. 2 d) after implant bone insertion being connected with external ambient by internal canals (FIG. 2 c).

2. Combined fixation bone implant, in accordance with claim 1, characterized in that the introduction of bone cement in the delimited cavities (FIG. 2 d) being carried through the orifice and respective canals (FIG. 2 c) by pressure through the use of an auxiliary device automatic or manual.

3. Combined fixation bone implant, in accordance with claim 1, characterized in that the bone cement being completely and only delimited by the volume cavities (FIG. 2 d) in the outer surface of implant (FIG. 2 e).

4. Combined fixation bone implant, in accordance with claim 1, characterized in that it assures the total removal of the bone cement during the movement of extraction of the implant, in the case of implant revision, being the bone cement delimited to the implant cavities (FIG. 2 d) in outer surface of implant and being the cavities perfectly aligned by side in axial direction of implant.

5. Combined fixation bone implant, in accordance with claim 1, characterized in that it assures the lowest damage of bone in the case of implant extraction, only to the bone that limit the cavities, being all the bone cement contained and delimited to the implant cavities (FIG. 2 d) in outer surface of implant and being the cavities perfectly aligned by side in axial direction of implant

6. Combined fixation bone implant, in accordance with claim 1, characterized in that it allows implant stability in the immediate postoperative period working the bone cement in cavity (FIG. 2 d) like a wedge glued to the bone, limiting the micromovements between implant and bone.

7. Combined fixation bone implant, in accordance with claim 1, characterized in that delimits the zones subject to thermal necrosis in the bone to the implant cavities (FIG. 2 d) zones, being the bone cement in contact with bone totally confined to the cavities (FIG. 2 d) in the outer surface of the implant.

8. Combined fixation bone implant, in accordance with claim 1, characterized in that the geometry providing zones of direct fixation with the bone surface and zones where the fixation between implant and bone are made by the bone cement delimited in implant cavities.
